(19) United States
(12) Patent Application Publication
(10) Pub. No.: US 2006/0088895 A1
(43) Pub. Date: Apr. 27, 2006

(54) Systems, methods and reagents for the detection of biological and chemical agents using dynamic surface generation and imaging

(76) Inventors: Bart J. Wanders, Oro Valley, AZ (US); Stuart A. Kushon, Santa Fe, NM (US)

Correspondence Address: Supervisor, Patent Prosecution Services;DLA PIPER RUDNICK GRAY CARY U.S. LLP, 1200 Nineteenth Street, N.W., Washington, DC, 20036-2412, US

(21) Appl. No.: 11/050,788

(22) Filed: Jan. 27, 2005

Related U.S. Application Data
(60) Provisional application No. 60/540,297, filed on Jan. 30, 2004.

(51) Int. Cl. G01N 33/554; C12M 1/34; G01N 33/569

(52) U.S. Cl. 435/7.32; 435/287.2

(57) ABSTRACT

Techniques for the sensitive detection of analytes which combine the benefits of solution/suspension phase assay formats and the simplicity of solid phase/lateral flow assays are described. The assays can be performed in the solution/suspension phase using magnetic microspheres as a solid support. Subsequently a magnetic separation can be performed to separate the bound analyte from the remainder of the solution. After a wash step, the fluorescence signal can be directly read from the magnetic particle surface. Portable biodetection systems which employ fluorescent polymer superquenching and methods for detecting bioagents therewith are also described.

This application claims priority from U.S. Provisional Application Ser. No. filed 60/540,297 filed Jan. 30, 2004. The entirety of that provisional application is incorporated herein by reference.

This application is related to U.S. Patent application Ser. No. 09/850,074, filed May 8, 2001, and U.S. patent application Ser. No. 10/621,311, filed Jul. 18, 2003. Each of these applications is incorporated herein by reference in its entirety.

### BACKGROUND

1. Technical Field

The present application relates generally to systems and methods for the detection of bioagents and, in particular, to portable biodetectors which employ fluorescence, to the use of such detectors for the detection of bioagents and to assay techniques and reagents which employ a magnetic solid phase.

2. Background of the Technology

Various pathogens may be present in the environment due to natural causes. In addition, the recent increase in bioterrorism threats throughout the world has made the early identification of harmful bioagents which have been intentionally introduced into the environment an increasingly urgent priority. While many assays for specific bioagents are available for use in specialized laboratories, there remains a need for robust and dependable systems and assays that may be carried out by relatively untrained personnel in the field. In particular, there is a continuing need for portable (i.e., hand-held) detection systems that can be used in the field to perform rapid, sensitive and selective assays for pathogens which may be present in the environment in a variety of forms including aerosols, powders or liquids.

### SUMMARY

According to a first embodiment, a cartridge is provided which comprises:

walls defining a detection reservoir; and

a fluid in the detection reservoir, the fluid comprising:
a particulate solid support which can be attracted by a magnetic field, wherein a surface of the particulate solid support comprises a receptor capable of binding a biological agent; and
a fluorescer which is capable of binding the biological agent; and

a port for introduction of a sample into the reservoir.

According to a second embodiment, a detection device is provided which comprises:

a housing adapted to receive a cartridge as set forth above;

an excitation light source adapted to impinge light on an interior surface of the detection reservoir of the cartridge; and

a detector adapted to detect fluorescent emissions from the interior surface of the detection reservoir of the cartridge.

According to a third embodiment, a kit for detecting the presence and/or amount of a biological agent in a sample is provided which comprises:

a first component comprising a particulate solid support which can be attracted by a magnetic field, wherein a surface of the particulate solid support comprises a receptor capable of binding the biological agent; and

a second component comprising a fluorescer capable of binding the biological agent when the biological agent is bound to the receptor.

According to a fourth embodiment, a method of detecting a biological agent in a sample is provided which comprises:

incubating the sample with a particulate solid support and a fluorescer in a reservoir of a container comprising walls defining the reservoir, wherein the particulate solid support can be attracted by a magnetic field, wherein a surface of the particulate solid support comprises a moiety capable of binding the biological agent and wherein the fluorescer comprises a moiety which is capable of binding the biological agent;

applying a magnetic field to the sample through a wall of the container such that solid support particles in the sample are attracted by the magnetic field thereby forming a surface adjacent the wall of the container;

impinging a light source on the surface formed by the solid support particles; and

detecting fluorescence emitted by the surface formed by the solid support particles;

wherein the detected fluorescence indicates the presence and/or amount of biological agent in the sample.

### BRIEF DESCRIPTION OF THE DRAWINGS

FIG. 1 is a representation of a biodetection device showing a biodetection cartridge being inserted therein.

FIG. 2 illustrates an assay wherein a fluorescent polymer and a bioreceptor are co-located on a solid support (i.e., a microsphere) showing how binding of an analyte quencher conjugate results in amplified superquenching of polymer fluorescence whereas binding of untagged analyte to the receptor results in no change in polymer fluorescence.

FIG. 3 illustrates an assay wherein a fluorescent polymer and a receptor for Staphylococcus Enterotoxin B (SEB) are co-located on a solid support and wherein the addition of an antibody tagged with a quencher results in amplified superquenching of fluorescence in the presence of the analyte (i.e., protein toxin SEB).

FIGS. 4A-4D illustrates a detection system which employs a magnetic solid phase and which involves dynamic surface generation via magnetic separation and imaging of the resulting surface.

FIG. 5 is a schematic depiction of an assay for a target biological agent employing a fluorescer and a magnetic particle each of which comprises a receptor capable of binding the target biological agent.

FIG. 6 is a schematic depiction of an assay for an antibody employing a fluorescer and a magnetic particle one of which comprises an antigen for the target antibody and the other of which comprises a receptor for the target antibody.

FIG. 7 is a schematic depiction of a FRET or superquenching assay for a target biological agent employing a third sensing component which includes a quencher/sensitized emitter with a recognition element for the target.

FIG. 8 is a schematic depiction of a FRET or superquenching assay for a target biological agent employing a magnetizable material which is embedded or coupled to a quenching material which may or may not act as a sensitized emitter.

FIG. 9 is a schematic depiction of various assays including: an assay wherein the addition or removal of phosphate groups by phosphatases and kinases is monitored (Reaction Scheme A); an assay wherein the cleavage of peptides by proteases or the ligation of DNA strands by a DNA Ligase is monitored (Reaction Scheme B); and an assay wherein the protein refolding process by an antibody for the natively folded protein is monitored (Reaction Scheme C).

FIG. 10 is a schematic depiction of an assay for a target nucleic acid involving DNA triplex formation employing first and second nucleic acid reagents each of which has affinity for the target and each of which comprises a biotin moiety, and a fluorescer and a magnetic particle each of which comprises a biotin binding protein.

FIG. 11A is a schematic depiction of a reaction cartridge which can be used in a detector.

FIG. 11B is a schematic depiction of a detector showing the reaction cartridge of FIG. 11A inserted therein.

FIG. 12 is a bar chart showing measured fluorescence as a function of the number of spores in a sample for Bacillus anthracis.

FIG. 13 is a bar chart showing measured fluorescence as a function of bioagent concentration for SEB.

FIG. 14 is a bar chart showing measured fluorescence as a function of bioagent concentration for ricin.

FIG. 15 is a bar chart showing measured fluorescence of samples containing various interferents compared to samples containing the interferent and Bacillus anthracis spores.

FIG. 16 is a bar chart showing measured fluorescence of samples containing large concentrations of bacillus spores other than Bacillus anthracis illustrating that none of these samples produced a positive signal in the Bacillus anthracis assay.

FIG. 17 is a bar chart showing measured fluorescence of samples containing various interferents compared to samples containing the interferent and ricin.

FIGS. 18A-18D are schematic depictions of an assay wherein: spores are mixed with magnetic particles and a fluorescent tag both of which can bind to a target biological agent (FIG. 18A); the magnetic particles and the fluorescent tag bind spores during mixing and incubation (FIG. 18B); the solution is magnetized resulting in bound and unbound magnetic material being attracted to the magnetized surface (FIG. 18C); and unbound fluorescent tag remaining in the solution is washed away (FIG. 18D).

### DETAILED DESCRIPTION

A portable (e.g., hand-held) autonomous instrument that can be used to detect bioagents (e.g., bacteria, toxins, viruses) in air, water or swabs from various surfaces is provided. According to one embodiment, detection can be accomplished in five minutes or less. The detector can comprise an alarm which signals the presence of the bioagent. Potential users of the device include emergency responders and hospital triage personnel. The biodetector device requires minimal technical expertise for operation and can detect and identify multiple agents with low cases of false positives and false negatives.

Several assay formats can be used in the biodetector device. Exemplary assay formats include solid phase (e.g., microsphere based) assays. Solid phase assays can be used, for example, to detect proteins and small molecule toxins. These assays do not require chemical or physical modification of the analyte (e.g., toxin) being detected and therefore permit detection of the analyte as it naturally exists in biological samples. Although solid phase assays are described above, assay formats employing soluble reagents can also be used.

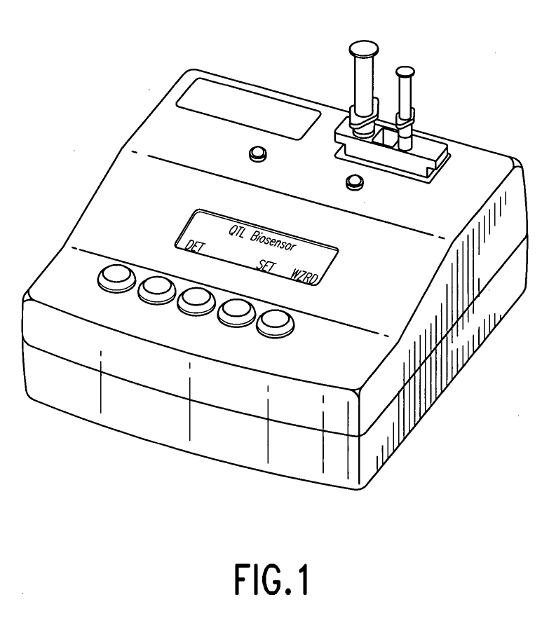

The assay steps can be carried out with a single-use, disposable cartridge. Such a device can be used by minimally trained operators with little likelihood of operator-introduced errors. An exemplary portable biodetection device is shown in FIG. 1.

Samples can be introduced into the cartridge using a disposable pipette. The sample volume can, for example, be 50 mL. A plunger in the cartridge can be used to generate liquid flows to complete the assay.

A biodetection system comprising a biodetector, one or more cartridges, and one or more positive and/or negative controls is also provided. The system controls can be used to insure the proper functioning of the biodetector.

Assays for various classes of biological and chemical agents are provided. Exemplary biological agents include, but are not limited to, bacteria (e.g., Bacillus anthracis), toxins (e.g., Staphylococcal enterotoxin B), and viruses (e.g., influenza). The bacterial agent may be sporulated. For example, detection cartridges for Bacillus anthracis and Staphylococcal enterotoxin B are provided. Other exemplary agents which can be detected are chemical and biological agents including sporulated bacteria, vegetative bacteria, viruses, protein toxins, proteases, choking agents, nerve agents, blister agents, and drugs of abuse. Specific examples of biological and chemical agents which can be detected include: Botulinum Toxins A, B, and E; Q-fever; plague (Yersinia Pestis); Vaccinia/Small Pox; Sarin Gas; Phosgene; VX Gas; and cocaine. In addition, assays can be generated for the detection of enzymes, enzymatic activity, nucleic acids (e.g., DNA), antibodies and small molecules such as caffeine and cocaine. Specific applications include assays for Bacillus anthracis, Staphylococcal enterotoxin B (SEB), Ricin toxin and a spore coat glycoprotein.

QTL Bioagent Detection

A first approach involves the use of a solid support (e.g., microspheres) containing a receptor for a target analyte (e.g., an SEB receptor such as an antibody or peptide receptor specific for SEB). For this approach, the solid support does not comprise a fluorescer. Once the analyte is exposed to the solid support bearing the receptor, a fluorescer comprising a moiety which binds the analyte (e.g., SEB-antibodies containing a highly fluorescent tag such as a polymer or other highly absorbing and fluorescent ensemble) can be bound to analyte captured on the solid support. The measurement of fluorescence intensity from the bound fluorescer provides a quantitative index of the analyte. This approach can be used to provide a sensitive, specific and quantitative assay for bioagents including, but not limited to, Bacillus anthracis, and SEB.

Assays employing amplified superquenching or Fluorescence Resonance Energy Transfer (i.e., FRET) are also provided. Moreover, polymers containing a series of chromophores which are either linked together via conjugation (i.e., conjugated polymers) or pendant and in close proximity on a non-conjugated polymer backbone (i.e., dye pendant polymers) exhibit a fluorescence emission that is altered from the fluorescence of an isolated monomer chromophore or dye. It has been shown that the fluorescence from these polymers is subject to an amplified response (i.e., superquenching) when the polymer is exposed and associates with very small amounts of certain energy or electron transfer quenchers. [1-3, 6] Thus, for a polymer consisting of a few hundred to several thousand units or chromophores per molecule, only a few molecules of a molecular quencher could “turn off” or quench the fluorescence from the entire polymer. This amplified quenching or superquenching of fluorescence is thus very much akin to the turning off of an entire string of Christmas tree lights when a single bulb is removed or burned out. Assays which employ amplified superquenching have been developed for a number of biological targets. [1, 7-9] These biodetection assays are based on fluorescence of polymers and polymer ensembles and their unique high sensitivity to fluorescence quenching by energy transfer or electron transfer quenchers.

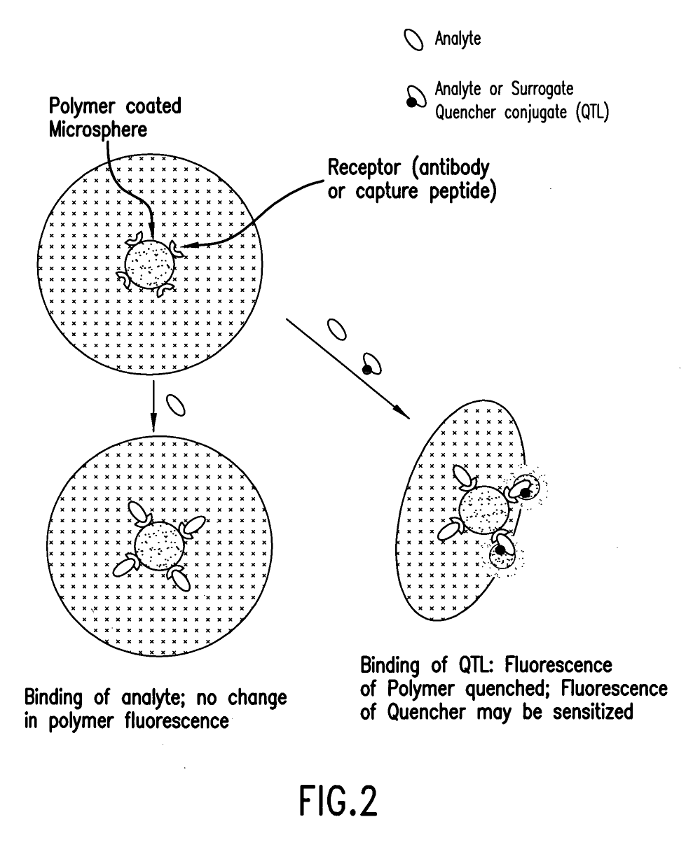

An assay wherein a fluorescent polymer and a bioreceptor are co-located on a microsphere or other solid support is shown in FIG. 2. As can be seen from FIG. 2, binding of the analyte to the receptor results in no change in polymer fluorescence whereas binding of an analyte quencher conjugate (e.g., a bioconjugate comprising a quencher, a tether, and a ligand for the receptor) results in amplified superquenching of the polymer fluorescence.

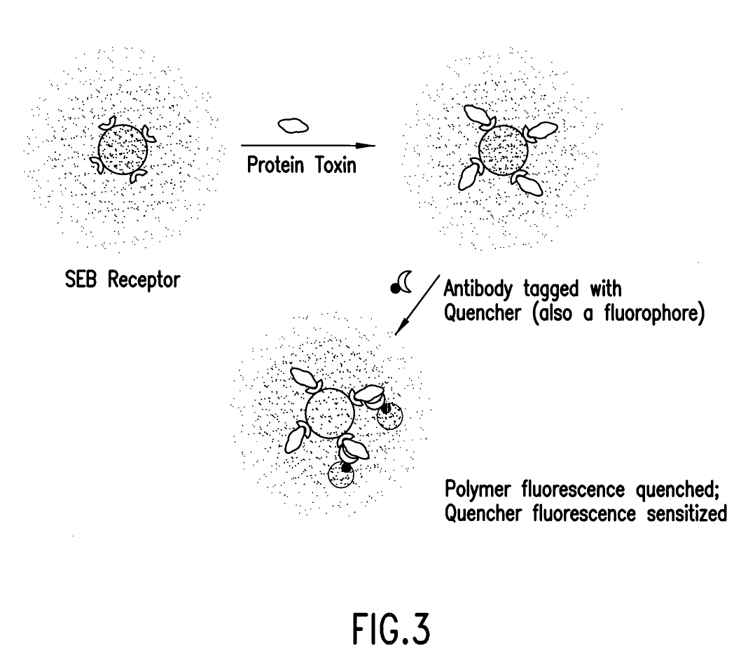

According to a second approach, a fluorescent polymer and a receptor for a bioagent (e.g., SEB or BT) are co-located on a solid support (e.g., a microsphere). The polymer and receptor can be conjugated to the support using known techniques. [1-5, 7-10] An assay of this type is shown in FIG. 3.

The receptor can be an antibody (e.g., a biotinylated antibody anchored to the support by biotin binding protein association) or a molecular receptor (for example, a biotinylated peptide). For SEB, commercial antibodies can be used or a a biotinylated peptide that binds to SEB can be synthesized. It has been shown that the polyclonal antibody binds solid support anchored SEB. In the first stage of this “sandwich assay”, the SEB analyte is captured on the microspheres. In the next stage, a second antibody, that has been functionalized with an energy transfer acceptor for the fluorescent polymer is exposed to the beads forming a “sandwich” with the anchored SEB, resulting in both quenching of the polymer fluorescence and sensitization of the acceptor fluorescence (at a different, longer wavelength). Either or both the polymer fluorescence and energy acceptor fluorescence may be monitored and the ratio will provide a quantitative measurement of the SEB level.

Dynamic Surface Generation and Imaging

In some applications, the target material is relatively large (e.g., nano or microparticles as opposed to small protein toxins). For these applications, the use of superquenching as described above may not be effective due to distance constraints inherent to the energy transfer mechanism. Accordingly, a detection technique is provided which combines the benefits of a solution/suspension phase assay format and the simplicity of a solid phase/lateral flow assay. This technique is suitable for several different assay types including, but not limited to, sandwich and competition formats.

When using this approach, the assay can be performed in the solution/suspension phase using a magnetic solid support (e.g., magnetic microspheres). Subsequently a magnetic separation can be performed to separate the bound analyte from the remainder of the solution. In this manner, a surface comprising magnetic particles is formed. After a wash step, the fluorescent signal can be directly read from the surface of magnetic particles instead of resuspending the particles and detecting fluorescence in solution.

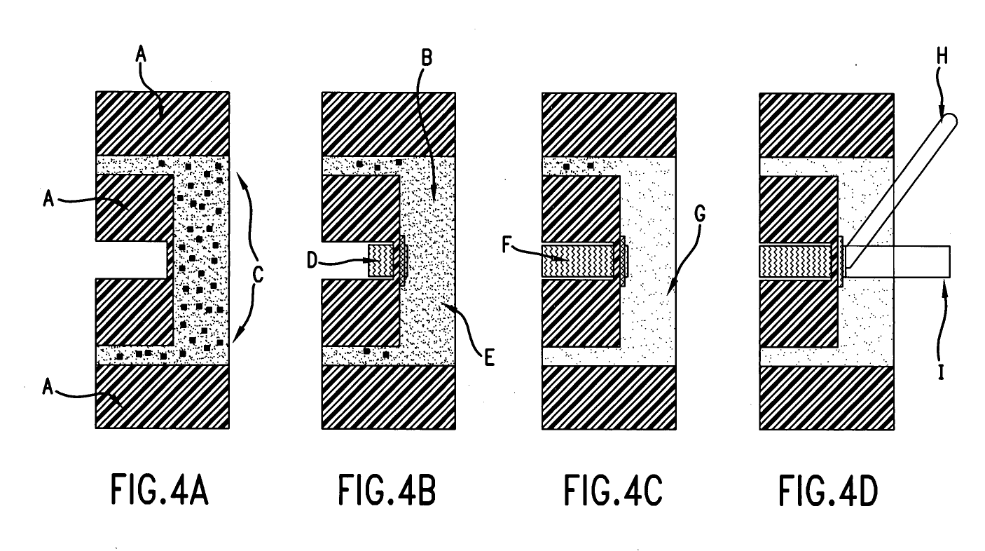

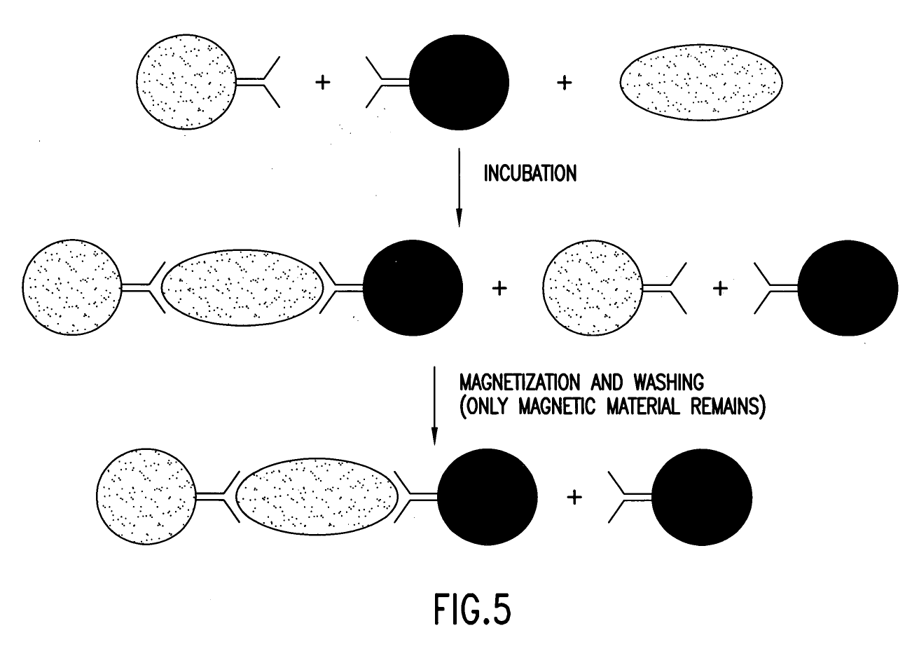

FIG. 4 illustrates a detection system and an assay involving dynamic surface generation and imaging. As shown in FIG. 4A, a cartridge frame (A) defines a detection reservoir containing magnetic microparticles dispersed in a tagging solution (B). The magnetic microparticles may be bound directly or indirectly to a fluorescent tag (C). The tagging solution is present in excess. As shown in FIG. 4B, a first magnetic field (D) is applied to generate a surface coated with magnetic particles (E) from the detection reservoir. After the surface has been formed, a second magnetic field (F) stronger than the first magnetic field is applied in preparation for a wash step to prevent dislodging the coating of magnetic particles. The assay can also be carried out with a single magnetic field strength. This step is shown in FIG. 4C. As the wash occurs, the tagging solution is replaced in the detection reservoir by a wash solution (G). Once the tagging solution has been removed, the surface or coating of magnetic particles can be imaged. As shown in FIG. 4D, during imaging, an excitation light source (H) is focused on the coating of magnetic particles while the emitted fluorescent light from the surface of the coating (I) is collected as a signal.

Binding of the fluorescent tag to the magnetic microparticles may occur, for example, in the presence of an analyte. For example, the fluorescent tag can be conjugated to a moiety which binds to the analyte. The analyte in the sample, in turn, can bind to a receptor on the surface of the magnetic microparticle. Therefore, magnetic microparticles become fluorescently tagged when analyte is present in the sample. Accordingly, the presence of analyte in the sample results in increased fluorescence.

Alternatively, the tagging solution may comprise fluorescent labeled analyte or analyte surrogate. When sample is added to the reservoir, analyte in the sample competes with the labeled analyte for analyte binding sites on the magnetic particles. The presence of analyte in the sample therefore results in reduced fluorescence.

One benefit of the above described dynamic surface generation and imaging technique is that the use of a solution/suspension phase ensures optimal kinetic conditions, while the formation of the magnetic particle coating concentrates the analyte resulting in a significant improvement in assay sensitivity. Another benefit of this technique is that the surface is created dynamically and does not exhibit the same non-specific binding problems often encountered in lateral flow assays which commonly utilize nylon and nitrocellulose membranes.

Highly sensitive systems and assays for the detection of pathogens, including protein toxins and microbes, are described herein. In particular, a handheld biosensor is provided which allows for rapid, on-site detection of dangerous bio-agents. Due to the simplicity and robustness of the chemistries employed for detection, these technologies can be easily applied to new biothreat agents as they arise.

Exemplary Assay Formats

An exemplary application of dynamic surface generation and imaging is a sandwich immunoassay wherein the fluorescer and the magnetizable material each comprise a receptor. Exemplary receptors include, but are not limited to, antibodies (e.g., monoclonal, polyclonal, single chain or antibody fragments), oligomeric aptamers (e.g., DNA, RNA, synthetic oligonucleotides), sugars, lipids, peptides, functional group binding proteins (e.g., biotin binding proteins, phosphate binding proteins), DNA, RNA, synthetic oligonucleotides, metal binding complexes, or any natural or synthetic molecule or complex with specific affinity for another molecule or complex. Moreover, the receptors can have specific affinity for a particular target material (e.g., chemical or biological agents). Upon generation of a fluorescer-target-magnetizable material complex, the solution can be magnetized and washed yielding a pellet which contains fluorescer only in the event that the target was present in the sample. An assay of this type is illustrated schematically in FIG. 5.

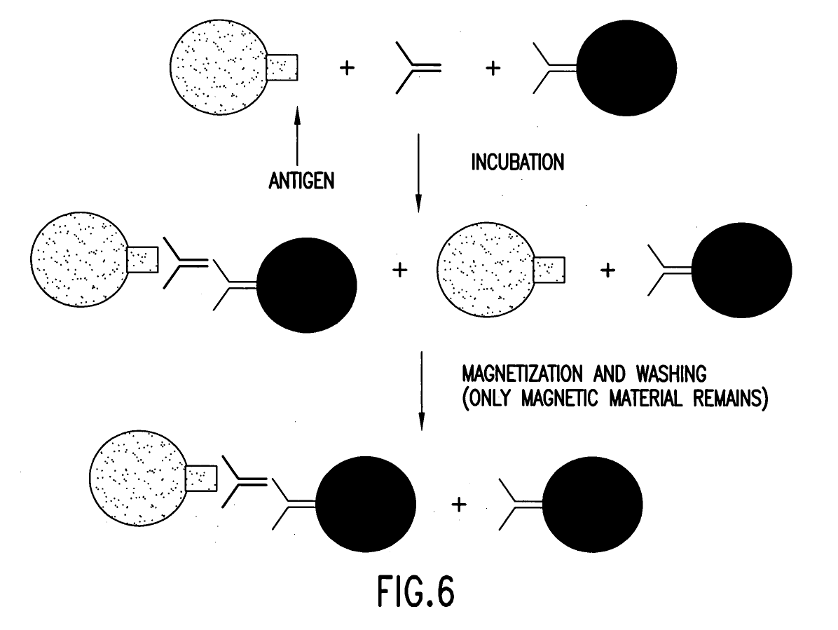

Another exemplary direct detection strategy is an antibody titer assay where either the fluorescer or the magnetizable material comprises an antigen for an antibody of interest. In this embodiment, the sensing material to which the antigen is not bound (i.e., either the fluorescer or the magnetizable material) can be linked to an antibody specific to antibodies generated by the animal species that generated the antibody of interest. When a sample in which the antibody of interest is present is incubated with a solution comprising these sensing materials, the antibody of interest can form a complex with the magnetic material and the fluorescer. As a result, a fluorescent signal can be detected in a dynamic surface generation and imaging assay when antibody of interest is present in the sample. An assay of this type is illustrated schematically in FIG. 6.

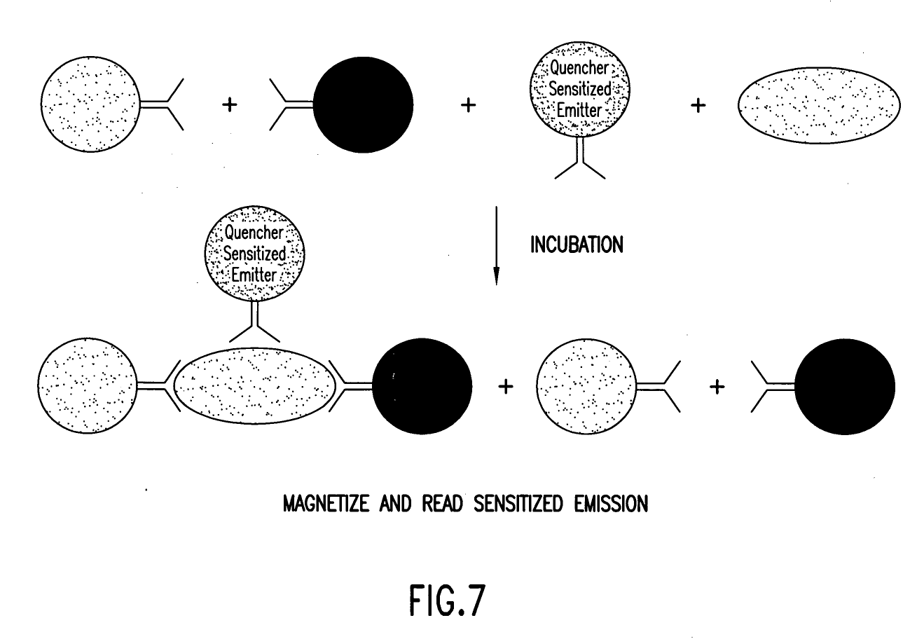

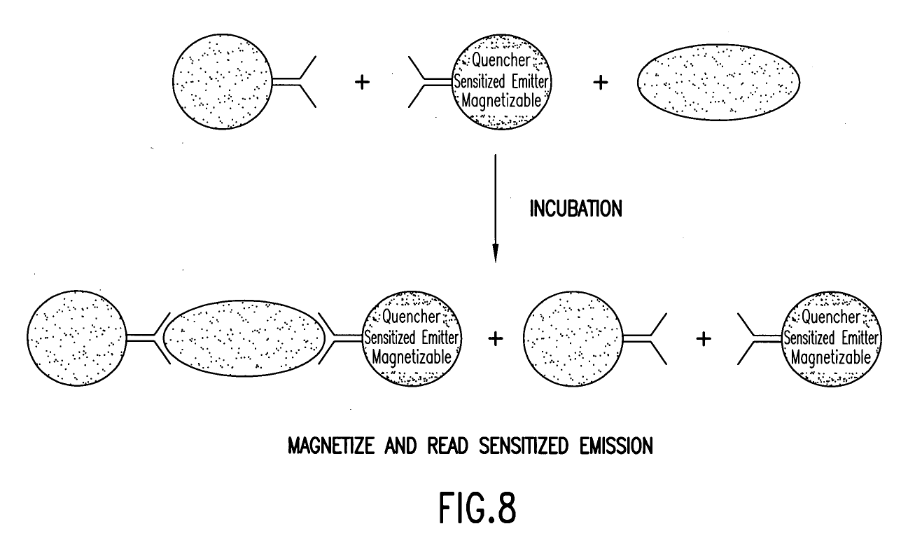

The technology depicted in FIG. 5 can be modified to either a FRET or superquenching based application using one of two routes. The first, involves the addition of a third sensing component comprising a quencher which may or may not be a sensitized emitter with a recognition element for the target as shown in FIG. 7. In the second route, the magnetic material comprises a quencher (e.g., an embedded or coupled quencher) which may or may not act as a sensitized emitter as shown in FIG. 8. In these sensitized emission routes, a wash step is not necessary to resolve the signal. If only a quencher is used, however, a wash step can be used to reduce background due to unbound fluorescer.

Alternative applications include monitoring chemical or biological changes such as structural modifications. Various exemplary assay formats are described below.

Addition or Removal of Chemical or Biological Moieties

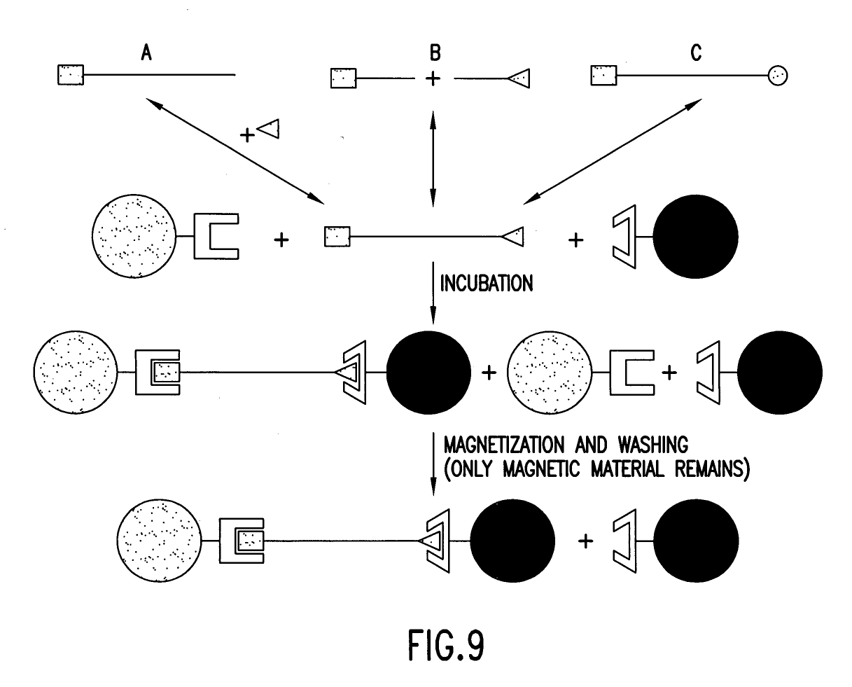

An example of this type of application is monitoring the addition or removal of phosphate groups by phosphatases and kinases. Exemplary starting materials include a biotinylated peptide with a site for phosphorylation, a fluorescer with a covalently linked biotin binding protein (e.g., avidin) and a magnetizable material with covalently linked phosphate binding protein. An assay of this type is shown schematically in reaction scheme A of FIG. 9.

Covalent/Non-Covalent Complexation/Attachment or Dissociation/Bond-Breaking

An exemplary application of this type of assay is monitoring the cleavage of peptides by proteases or the ligation of DNA strands by a DNA ligase. Exemplary starting materials include a peptide comprising two biotins with a protease recognition between, a fluorescer comprising a biotin binding protein (e.g., avidin), and magnetic material comprising a biotin binding protein (e.g., avidin). The biotin binding protein can be covalently linked to the fluorescer and/or the magnetic material.

An assay of this type which involves complexation/attachment is shown schematically in reaction scheme B of FIG. 9 wherein a complexation/attachment event results in the formation of a fluorescer/magnetic material complex.

Chemical or Biological Modifications or Folding

An application of this type involves the monitoring of a protein refolding process by an antibody for the natively folded protein. Applications of this type are not limited, however, to a refolding process, but also include any detectable chemical or biological moieties. Exemplary starting materials include an unfolded protein with a covalently linked biotin, a fluorescer comprising a biotin binding protein (e.g., avidin) which can be covalently linked to the fluorescer, and a magnetic material comprising an antibody for the natively folded protein.

An assay of this type is shown schematically in reaction scheme C of FIG. 9 wherein folding (indicated in the figure by the conversion of the ● to the ▴) results in recognition of the protein by the antibody linked to the magnetic material thereby resulting in the formation of a fluorescer/magnetic material complex.

The Generation of Complexes that Contain Multiple Receptor Sites

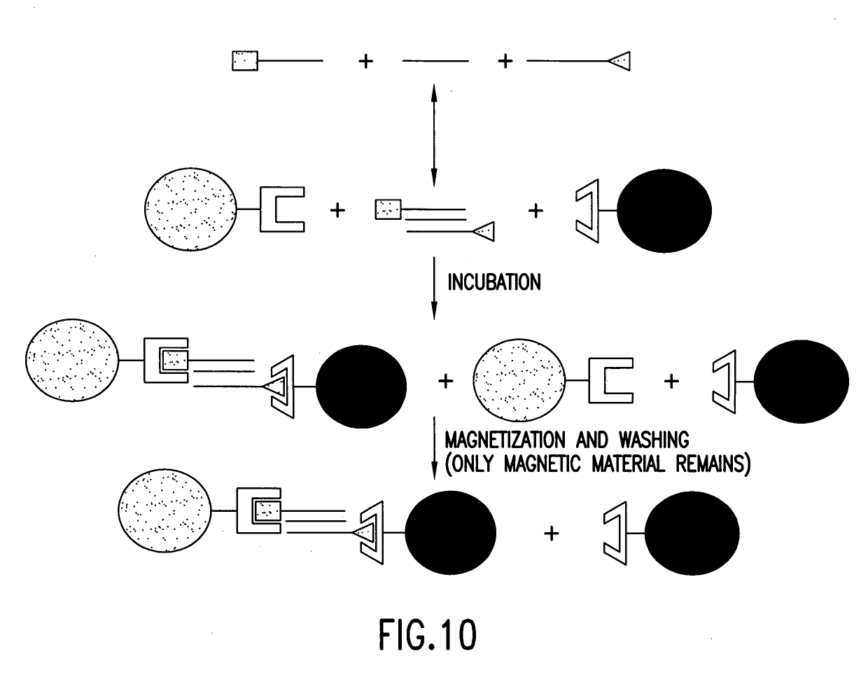

Exemplary assays include assays in which complexes containing multiple receptor sites are generated. An exemplary assay of this type involves a DNA triplex formation. Exemplary starting materials include first and second nucleic acids each of which has affinity for a target nucleic acid and each of which also comprises a biotin moiety, and a fluorescer and a magnetic material each comprising a biotin binding protein (e.g., avidin). The biotin binding protein can be covalently linked to the fluorescer and/or the magnetic material with. An assay of this type is shown schematically in FIG. 10.

The above described assays and formats are generally applicable to any system wherein a surface of magnetic particles (i.e., a pellet) is generated that can be focused upon with both an excitation source and a detector. For example, the assay can be performed on a plate reader as set forth below. First, the samples in the plate are magnetized through the use of a rack that places a magnet below the wells of the plate and allows for the formation of magnetic pellets in specific locations on the bottom of the wells. The samples are then washed. A light source of the plate reader and the detector of the plate reader are then focused optically so that the pellets that are formed are excited and monitored for fluorescence output.

The above strategy can be used in any chip based application where a magnet can be oriented to form a pellet and a light source and a detector can be focused to excite and collect the emission from that pellet.

### EXAMPLES

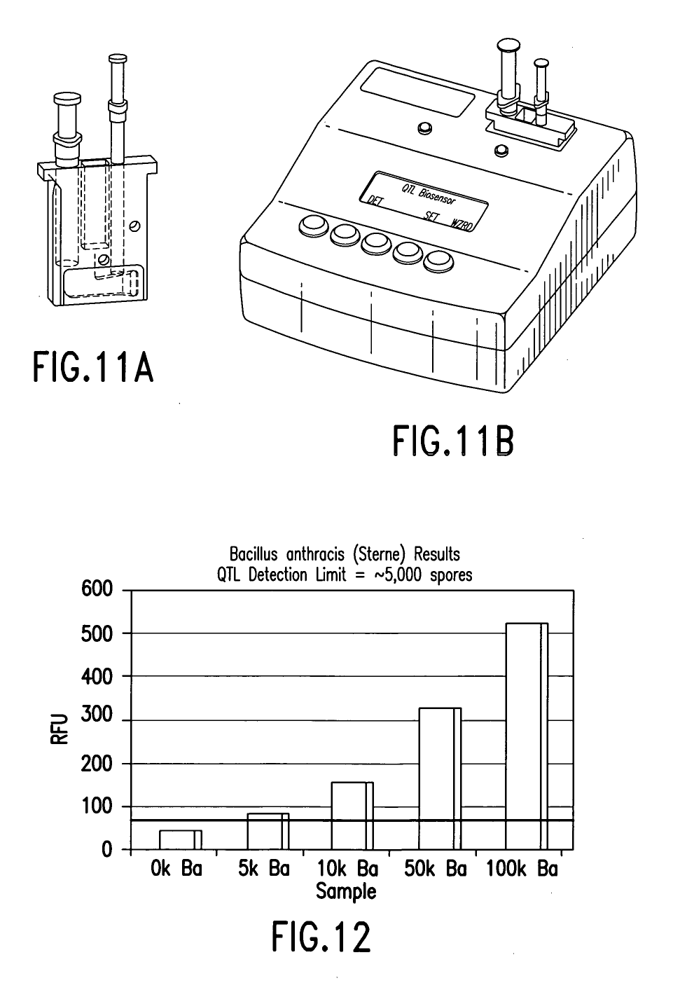

Detection and quantification assays for Bacillus anthracis, Ricin (Castor Bean Toxin), and Staphyloccocal Enterotoxin B have been developed using a dynamic surface generation and imaging method. An apparatus for performing this assay is shown in FIGS. 11A and 11B. As shown in FIG. 11A, the assay can use a cartridge that is preloaded with sensing materials (e.g., fluorescer with receptor for bioagent, and magnetic material with a receptor for the bioagent). These sensing materials can be prepared in a dried form for long term storage. A washing syringe containing a wash solution (the larger syringe shown in FIG. 11A) can be inserted in the cartridge. The sample containing the material of interest for testing can be prepared in a sampling solvent either through a swabbing kit or dilution, and then collected into the sampling syringe (the smaller syringe shown in FIG. 11A). The sample syringe is then inserted into the cartridge. The sample is then added to the sensing reagents by depressing the barrel of the sampling syringe. The cartridge can then be shaken for 1 minute (this step is less important for toxin assays than for spore assays). The cartridge is then inserted into the detector unit for a 2 minute incubation period as shown in FIG. 11B. During this time the magnetic material is magnetized and generates a surface which displays the fluorescer in the presence of the biological agent of interest. After excitation and collection of emitted fluorescence the result (e.g., target present or no target present) is available for the user. Excitation and collection of emitted fluorescence can be accomplished in 5 seconds. The total time required for an assay can be approximately 3.5 minutes.

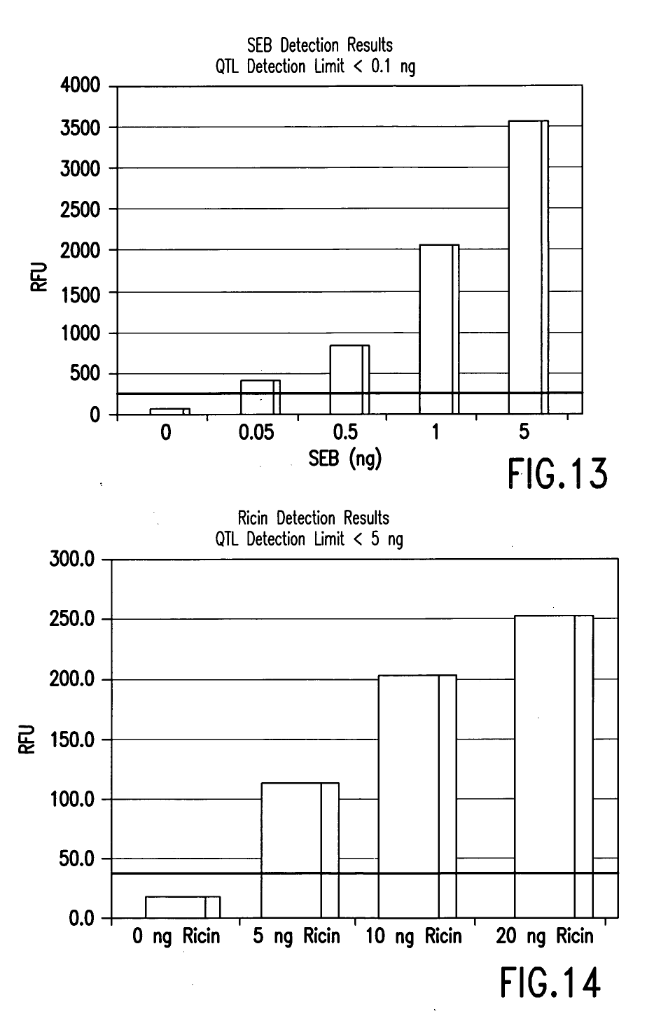

Data which have been collected using dynamic surface generation and imaging are shown in FIGS. 12-16.

Limits of detection were determined form the data shown in FIGS. 12-14 as follows:

| Target | Limit of Detection |
|---|---|
| Bacillus Anthracis | approximately 5,000 spores; |
| Ricin | <5 ng; |
| SEB | <0.1 ng.. |

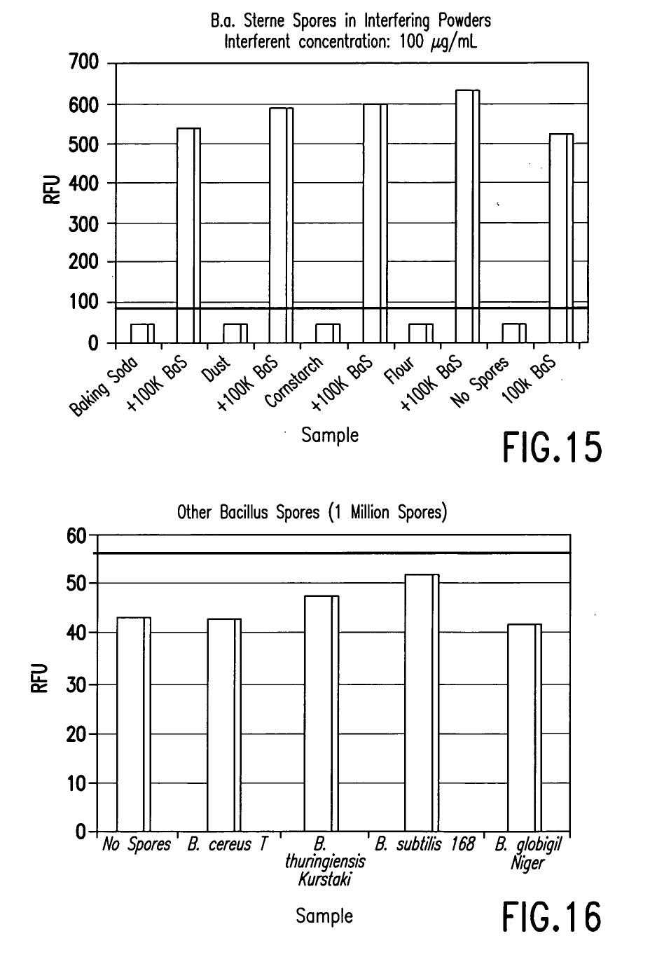

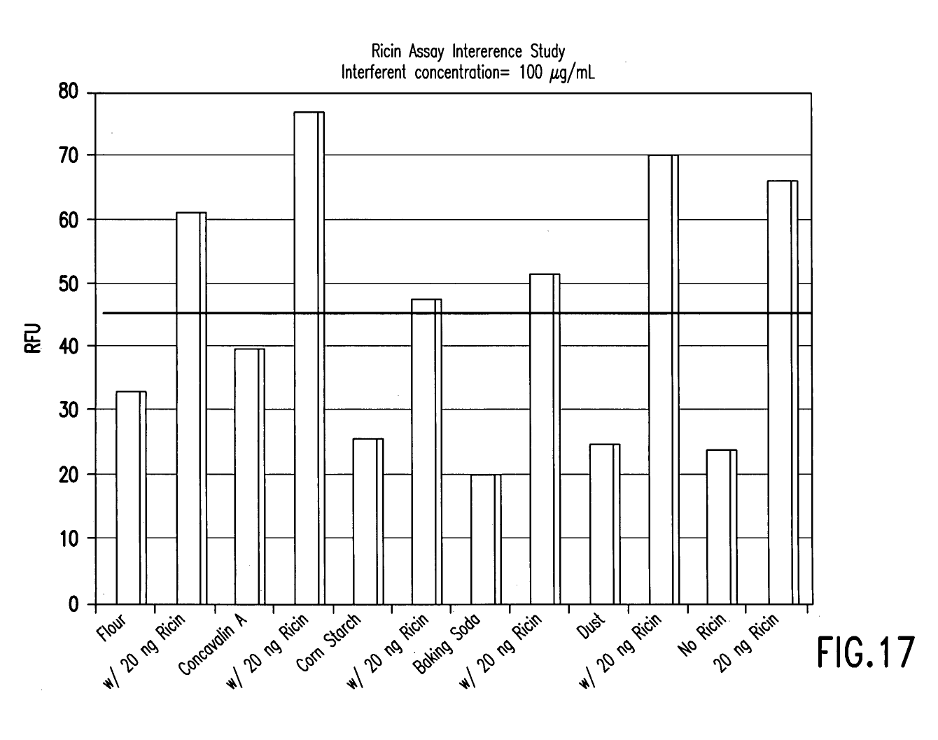

Interferents of baking soda, corn starch, flour, and Arizona test dust have been tested as shown in FIGS. 15 and 17 and were determined to have limited effects of the assay and did not result in a positive signal (i.e., a false positive). Signal variation, however, can occur in the presence of some interferents. However, none of the materials tested completely inhibits the assay at the concentrations used (i.e., at an interferent concentration of 100 μg/mL, which is 100,000-fold the concentration of the toxin analytes used).

Nearest neighbor spores to Bacillus anthracis have also been tested in the Bacillus Anthracis assay format as shown in FIG. 16 and none of these spores showed positive signals even at levels of one million spores per assay.

Thus, the assays generated by dynamic surface generation and imaging are both sensitive and specific. Furthermore, the mixture of sensing reagents is capable of generation multiplexed assays for multiple bioagents. This can be performed in a number of ways, but the most simple are mixing two sensors together, or generating a multisensor by putting multiple receptors of the fluorescer and magnetizable material. The later of these two routes can be a single color assay where the result is either target A or B is present, while the former route (multiple sensors) can be a multi-color assay where if A is present one color of fluorescence is present, and if B is present another color is present. In this embodiment, the fluorescers are of different colors.

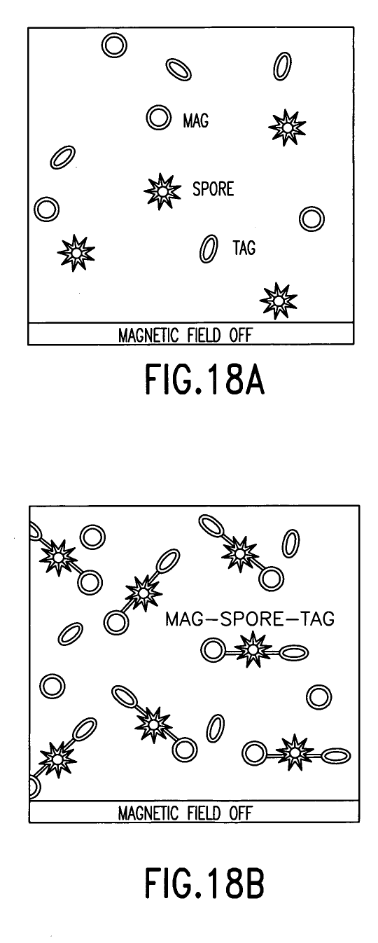

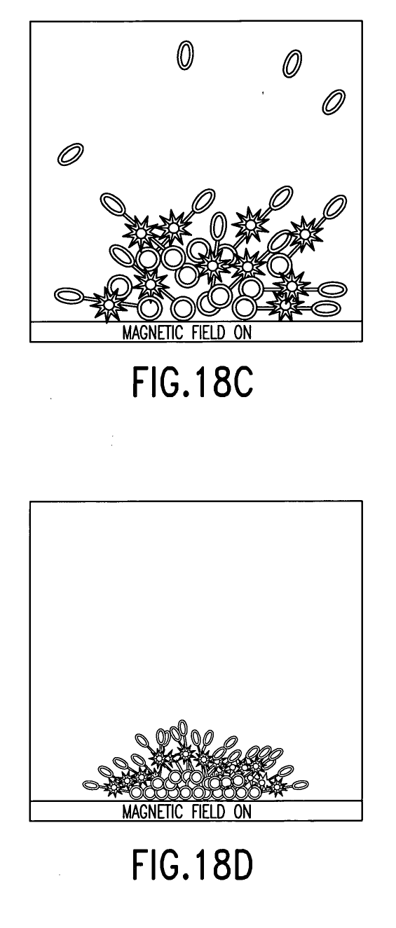

An exemplary assay format is illustrated in FIGS. 18A-18D. As shown in FIG. 18A, spores are mixed with QTL Sensing Solution comprising magnetic microspheres and a fluorescent tag both of which can bind to a target biological agent (spore shown). As can be seen from FIG. 18B, the sensing materials (i.e., the magnetic microspheres and the fluorescent tag) can bind spores during mixing and incubation. The solution is then magnetized as shown in FIG. 18C. Application of the magnetic field results in the bound and unbound magnetic material being attracted to the surface. Unbound fluorescent tag remaining in solution can then be washed away as shown in FIG. 18D. The presence of fluorescence emitted by the excited surface indicates the presence and/or amount of the target biological agent in the sample.

While the foregoing specification teaches the principles of the present invention, with examples provided for the purpose of illustration, it will be appreciated by one skilled in the art from reading this disclosure that various changes in form and detail can be made without departing from the true scope of the invention.

### REFERENCES CITED

[1] Chen, L., D. W. McBranch, H.-L. Wang, R. Helgeson, F. Wudl, and D. G. Whitten, “Highly sensitive biological and chemical sensors based on reversible fluorescence quenching in a conjugated polymer”, Proceedings of the National Academy of Sciences of the United States of America, 96, pp. 12287-12292 (1999).
[2] Chen, L., D. W. McBranch, R. Wang, and D. Whitten, “Surfactant-Induced Modification of Quenching of Conjugated Polymer Fluorescence by Electron Acceptors: Applications for Chemical Sensing”, Chemical Physics Letters, 330, pp. 27-33 (2000).
[3] Chen, L., S. Xu, D. McBranch, and D. Whitten, “Tuning the Properties of Conjugated Polyelectrolytes through Surfactant Complexation”, Journal of the American Chemical Society, 112(28), pp. 9302-9303 (2000).
[4] Jones, R. M., L. Lu, R. Helgeson, T. S. Bergstedt, D. W. McBranch, and D. Whitten, “Building Highly Sensitive Dye Assemblies for Biosensing from Molecular Building Blocks”, Proceedings of the National Academy of Sciences of the United States of America, 98(26), pp. 14769-14772 (2001).
[5] Lu, L., R. M. Jones, D. McBranch, and D. Whitten, “Surface-Enhanced Superquenching of Cyanine Dyes as J-Aggregates on Laponite Clay Nanoparticles”, Langmuir, 18(20), pp. 7706-7713 (2002).
[6] Lu, L., R. Helgeson, R. M. Jones, D. McBranch, and D. G. Whitten, “Superquenching in Cyanine Pendant Poly(L-lysine) Dyes: Dependence on Molecular Weight, Solvent, and Aggregation”, Journal of the American Chemical Society, 124(3), pp. 483-488 (2002).
[7] Kushon, S. A., K. Bradford, V. Marin, C. Suhrada, B. A. Armitage, D. McBranch, and D. Whitten, “Detection of Single Nucleotide Mismatches Via Fluorescent Polymer Superquenching”, Langmuir, 19, pp. 6456-6464 (2003).
[8] Kushon, S. A., K. D. Ley, K. Bradford, R. M. Jones, D. McBranch, and D. Whitten, “Detection of DNA Hybridization via Fluorescent Polymer Superquenching”, Langmuir, 18(20), pp. 7245-7249 (2002).
[9] Kumaraswamy, S., T. Bergstedt, X. Shi, F. Rininsland, K. Achyuthan, S. Kushon, K. Ley, W. Xia, D. McBranch, and D. Whitten, “Fluorescent Conjugated Polymer Superquenching Facilitates Highly Sensitive Detection of Proteases”, Proceedings of the National Academy of Sciences of the United States of America, 101, 7511-7515 (2004).
[10] Cooper, M. A., “Optical Biosensors in Drug Discovery”, Nature Reviews, 1, pp. 515-528 (2002).

What is claimed is:

1. A cartridge comprising:
walls defining a detection reservoir; and
a fluid in the detection reservoir, the fluid comprising:
a particulate solid support which can be attracted by a magnetic field, wherein a surface of the particulate solid support comprises a receptor capable of binding a biological agent; and
a fluorescer which is capable of binding the biological agent; and
a port for introduction of a sample into the reservoir.

2. The cartridge of claim 1, further comprising a plunger adapted to generate a flow of the liquid in the detection reservoir.

3. The cartridge of claim 1, wherein the fluorescer comprises a plurality of fluorescent species associated with one another such that a quencher is capable of amplified superquenching of fluorescence emitted by the fluorescer when associated therewith.

4. The cartridge of claim 1, wherein the biological agent is Staphylococcus Enterotoxin B, Botulinum Toxin, or Bacillus Anthracis.

5. The cartridge of claim 1, wherein the particulate solid support is a microsphere.

6. The cartridge of claim 1, wherein the particulate solid support comprises a quencher which is capable of quenching fluorescence emitted by the fluorescer when the particulate solid support and fluorescer are bound to the biological agent.

7. The cartridge of claim 6, wherein the quencher emits fluorescence.

8. The cartridge of claim 1, wherein the fluid in the detection reservoir further comprises a quencher capable of binding the biological agent when the biological agent is bound to the particulate solid support and the fluorescer; wherein the quencher is capable of quenching fluorescence emitted by the fluorescer when associated therewith.

9. A detection device comprising:
a housing adapted to receive a cartridge as set forth above;
an excitation light source adapted to impinge light on an interior surface of the detection reservoir of the cartridge; and
a detector adapted to detect fluorescent emissions from the interior surface of the detection reservoir of the cartridge.

10. The detection device of claim 9, further comprising an indicator which is adapted to signal when the biological agent is present in the detection reservoir.

11. The detection device of claim 9, wherein the indicator is an alarm which sounds when biological agent is present in the detection reservoir.

12. The detection device of claim 9, further comprising a magnetic field generator adapted to apply a magnetic field to the fluid in the detection reservoir through a wall of the container.

13. The detection device of claim 12, wherein the magnetic field generator can generate magnetic fields of at least two different strengths.

14. The detection device of claim 9, further comprising a port for removing fluid from the reservoir.

15. A kit for detecting the presence and/or amount of a biological agent in a sample comprising:
a first component comprising a particulate solid support which can be attracted by a magnetic field, wherein a surface of the particulate solid support comprises a receptor capable of binding the biological agent; and
a second component comprising a fluorescer capable of binding the biological agent when the biological agent is bound to the receptor.

16. The kit of claim 15, wherein the particulate solid support is a microsphere.

17. The kit of claim 15, wherein the biological agent is Staphylococcus Enterotoxin B, Botulinum Toxin, or Bacillus Anthracis.

18. The kit of claim 15, further comprising:
a third component comprising a quencher capable of binding the biological agent when the biological agent is bound to the receptor and the fluorescer, wherein the quencher is capable of quenching fluorescence emitted by the fluorescer when associated therewith.

19. The kit of claim 18, wherein the fluorescer comprises a plurality of fluorescent species associated with one another such that the quencher is capable of amplified superquenching of fluorescence emitted by the fluorescer when associated therewith.

20. The kit of claim 18, wherein the quencher emits fluorescence.

21. The kit of claim 15, wherein the particulate solid support comprises a quencher which is capable of quenching fluorescence emitted by the fluorescer when the particulate solid support and fluorescer are bound to the biological agent.

22. The kit of claim 21, wherein the quencher emits fluorescence.

23. A method of detecting a biological agent in a sample comprising:
incubating the sample with a particulate solid support and a fluorescer in a reservoir of a container comprising walls defining the reservoir, wherein the particulate solid support can be attracted by a magnetic field, wherein a surface of the particulate solid support comprises a moiety capable of binding the biological agent and wherein the fluorescer comprises a moiety which is capable of binding the biological agent;
applying a magnetic field to the sample through a wall of the container such that solid support particles in the sample are attracted by the magnetic field thereby forming a surface adjacent the wall of the container;
impinging a light source on the surface formed by the solid support particles; and
detecting fluorescence emitted by the surface formed by the solid support particles;
wherein the detected fluorescence indicates the presence and/or amount of biological agent in the sample.

24. The method of claim 23, further comprising washing the surface formed by the solid support particles after applying a magnetic field and before impinging a light source.

25. The method of claim 24, further comprising increasing the strength of the applied magnetic field after applying a magnetic field and before washing.

26. The method of claim 23, further comprising:
incubating the sample with a quencher capable of binding the biological agent when the biological agent is bound to the particulate solid support and the fluorescer, wherein the quencher is capable of quenching fluorescence emitted by the fluorescer when associated therewith.

27. The method of claim 26, wherein the fluorescer comprises a plurality of fluorescent species associated with one another such that the quencher is capable of amplified superquenching of fluorescence emitted by the fluorescer when associated therewith.

28. The method of claim 26, wherein the quencher can emit fluorescence and wherein detecting comprises detecting fluorescence emitted by the quencher and, optionally, also detecting fluorescence emitted by the fluorescer.

29. The method of claim 26, wherein detecting comprises detecting fluorescence emitted by the fluorescer.

30. The method of claim 23, wherein the particulate solid support comprises a quencher which is capable of quenching fluorescence emitted by the fluorescer when the particulate solid support and fluorescer are bound to the biological agent.

31. The method of claim 30, wherein the quencher emits fluorescence.

32. The method of claim 23, wherein a surface of the particulate solid support comprises a second moiety which is capable of binding a second biological agent and wherein the fluorescer comprises a second moiety which is capable of binding the second biological agent when the second biological agent is bound to the particulate solid support.

33. The method of claim 23, further comprising:
incubating the sample with a second particulate solid support and a second fluorescer in the reservoir, wherein the particulate second particulate solid support can be attracted by a magnetic field, wherein a surface of the second particulate solid support comprises a moiety capable of binding a second biological agent and wherein the second fluorescer comprises a moiety which is capable of binding the second biological agent when the second biological agent is bound to the particulate solid support;
wherein fluorescence emitted by the second fluorescer can be distinguished from that emitted by the fluorescer;
wherein fluorescence emitted by the fluorescer indicates the presence and/or amount of biological agent in the sample and wherein fluorescence emitted by the second fluorescer indicates the presence and/or amount of second biological agent in the sample.
